(19) United States
(12) Patent Application Publication
(10) Pub. No.: US 2002/0146588 A1
(43) Pub. Date: Oct. 10, 2002

(54) Novel compound for red organic electroluminescent elements and devices

(76) Inventors: Liang-Jyi Chen, Taipei (TW); Wen-Kuo Weng, Taipei Hsien (TW); Chun-Neng Ku, Hsinchu (TW); Po-Yen Lu, Taoyuan Hsien (TW)

Correspondence Address: BIRCH STEWART KOLASCH & BIRCH, PO BOX 747, FALLS CHURCH, VA, 22040-0747, US

(21) Appl. No.: 09/855,649

(22) Filed: May 16, 2001

Related U.S. Application Data
(63) Continuation-in-part of application No. 09/810,242, filed on Mar. 19, 2001, now Pat. No. 6,427,116.

(51) Int. Cl. H05B 33/14; C07D 311/02

(52) U.S. Cl. 428/690; 428/704; 428/917; 313/504; 313/506; 549/381; 549/398; 549/407

(57) ABSTRACT

The present invention discloses a novel compound useful as the material for a red organic EL device. The novel compound is produced by connecting a benzene ring at the positions 2 and 3 of withdrawing group 2, 5-dimethyl-4-(2,2-dicyano)pyrane, and connecting a conjugated donating group at position 5. Using this compound, the EL emission is shifted to the red spectral region, and a higher purity in color for red EL elements is obtained. The synthesis of the compound is easy and the product yield is improved compared to the prior art. Moreover, the red organic EL devices fabricated using the compound have properties that conform with existing NTSC standards.

This application is a continuation-in-part of application Ser. No. 09/810,242 filed Mar. 19, 2001, now pending.

### BACKGROUND OF THE INVENTION

1. Field of the Invention

The present invention relates to a novel compound for organic electroluminescent (EL) elements and devices, and more particularly to a novel compound for red organic EL elements and devices.

2. Description of the Prior Art

Organic EL devices are known to be highly efficient and are capable of producing a wide range of colors. Useful applications such as flat-panel displays have been contemplated. Representatives of earlier organic EL devices are Gurnee et al U.S. Pat. No. 3,172,862, and Gurnee U.S. Pat.

No. 3,173,050. Typical organic emitting materials were formed of a conjugated organic host material and a conjugated organic activating agent having condensed benzene rings. Naphthalene, anthracene, phenanthrene, pyrene, benzopyrene, chrysene, picene, carbazole, fluorene, biphenyl, terphenyls, and 1,4-diphenyl butadiene were offered as examples of organic host materials.

The most recent discoveries in the art of organic EL device construction have resulted in devices having the organic EL medium consisting of extremely thin layers (<1.0 micrometer in combined thickness) separating the anode and cathode. The organic EL medium is herein defined as the organic composition between the anode and cathode electrodes. In a basic two-layer EL device structure, one organic layer is specifically chosen to inject and transport holes and the other organic layer is specifically chosen to inject and transport electrons. The interface between the two layers provides an efficient site for the recombination of the injected hole-electron pair and resultant electroluminescence.

At present, red EL materials are produced by doping. Its light emission is generally produced by energy transfer between the host material and the guest material. Patents such as U.S. Pat. No. 5,935,720 and European Pat. No. 0791849A1 have disclosed such materials. However, the degree of synthetic complexities of the common material used for red EL elements is high, consequently the yield loss is elevated. Hence, it is necessary to provide a material that is easy to synthesize. In addition, the material preferably has a high purity in color and has properties that conform with NTSC standards (maximum wavelength λₘₐₓ and CIE coordinate).

### SUMMARY OF THE INVENTION

The object of the present invention is to provide a novel compound that is a suitable material for red organic EL elements and devices.

Another object of the invention is to provide a material that is easy to synthesize and has a high purity in color for red organic EL elements and devices.

Another object of the invention is to provide an organic EL device that conforms with NTSC standards.

To achieve the above-mentioned object, the novel compound is produced by connecting a benzene ring at the positions 2 and 3 of a withdrawing group 2,5-dimethyl-4-(2,2-dicyano)pyrane, and connecting a conjugated donating group at position 5. By doing so, the EL emission is shifted to the red spectral region. Hence, a novel material that has a higher purity in color for red EL elements and devices is obtained.

### DETAILED DESCRIPTION OF THE INVENTION

The novel compound of the present invention is shown as the following formula:

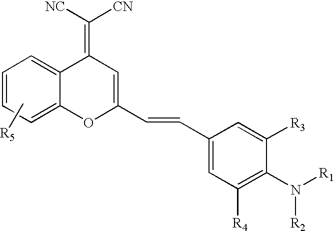

wherein, R₁ and R₂ are individually hydrogen, alkyl of from 1 to 20 carbon atoms, aryl, carbocyclic and other heterocyclic system, and R₃ and R₄ are individually hydrogen, alkyl of from 1 to 10 carbon atoms, and a branched or unbranched 5 or 6 member substituent ring connecting with R₁ and R₂ respectively; and R₅ is hydrogen, alkyl of from 1 to 10 atoms and a 5 or 6 member carboncyclic and other heterocyclic system connecting with benzenic ring; but when R₃₋₅ have no substitution groups present, R₁ and R₂ are hydrogen, alkyl of 2 to 20 carbon atoms, aryl, carbocyclic and other heterocyclic system.

In the above compound, examples of R₁ and R₂ are methyl, ethyl, propyl, n-butyl, —(CH₂)₄—, —(CH₂)₅—, aryl, such as phenyl, furyl, thienyl, pyridyl or other heterocyclic system; R₃ and R₄ are hydrogen, methyl, ethyl, propyl, n-butyl, i-propyl, t-butyl, sec-butyl, t-amyl and —(CH₂)₃—, —(CH₂)₄—, or heteroaryl, including phenyl furyl, thienyl, pyridyl and other heterocyclic system connecting with benzenic ring.

The synthetic procedure of the above compound is as follows: the mixture of 2-methyl-4-(2,2-dicyanomethylene)chromone, toluene, piperdine, acetic acid and a conjugated donating group is heated and refluxed for 18-20 hours. The mixture is then cooled to room temperature. After filtering, the mixture is washed with a small amount of toluene. Finally, the mixture is purified by sublimation.

Examples of the preferred conjugated donating group are such as 9-formyl-julolidine, 4,(N,N-dimethyl)anlinealdehyde, and 9-formyl-1-(1,1,7,7-tetramethyl)julolidine.

### BRIEF DESCRIPTION OF THE DRAWINGS

FIG. 1 is the graph illustrating brightness vs voltage for the EL device prepared in Embodiment 4.

FIG. 2 is the graph illustrating intensity vs wavelength for the EL device prepared in Embodiment 4.

The following examples exemplify the synthesis of the novel compound and the fabrication of EL devices using the same.

### EXAMPLE 1

Synthesis of Compound A

5 g of 2-methyl-4-(2,2-dicyanomethylene)chromone, 20 ml of toluene, 1.5 ml of piperdine, 1.5 ml of acetic acid and 6.5 g of 9-formyl-julolidine were placed in a 50 ml reaction vessel. The mixture was heated and refluxed for 18 hours. The mixture was then cooled to room temperature. After filtering, the mixture was washed with a small amount of toluene to obtain a product yield of 68%. Finally, the mixture was purified by sublimation. The melting temperature of the product is 236° C. ¹H-NMR: 8.88(1H, d, J=8.2 Hz), 7.73(1H, t, J=8.6 Hz), 7.43˜7.40(3H, m), 6.77(2H, br), 3.26(4H, t, J=5.8 Hz), 2.75(4H, t, J=4.6 Hz), 1.96(4H, t, J=5.4 Hz) ppm ∘ Mass: 393(M+2) ∘ IR: 2205, 1623, 1588, 1552, 1478, 1312, 1156, 769 cm⁻¹^{_{∘}}

Formula of compound A is:

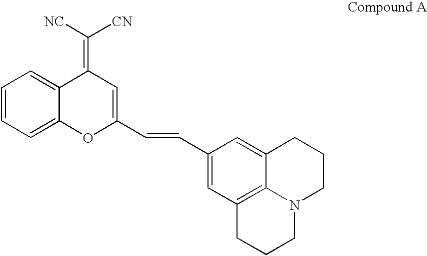

### EXAMPLE 2

Synthesis of Compound B

5 g of 2-methyl-4-(2,2-dicyanomethylene)chromone, 20 ml of toluene, 1.5 ml of piperdine, 1.5 ml of acetic acid and 5 g of 4-(N,N-dimethyl)anlinealdehyde were placed in a 50 ml reaction vessel. The mixture was heated and refluxed for 18 hours. The mixture was then cooled to room temperature. After filtering, the mixture was washed with a small amount of toluene to obtain a product yield of 78%. Finally, the mixture was purified by sublimation. The melting temperature of the product is 270° C. ¹H-NMR: 8.89(1H, d, J=4.8 Hz), 7.72(1H, t, J=7.6 Hz), 7.68˜7.39(6H, m), 7.03(1H, br), 6.67(1H,S), 6.62(1H, d, J=15.6 Hz), 3.08(6H, s) ppm ∘ Mass: 393(M⁺) ∘ IR: 2199, 1627, 1591, 1552, 1166, 979, 811 cm^{31 1}∘

Formula of compound B is:

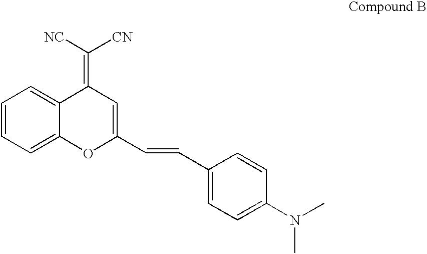

### EXAMPLE 3

Synthesis of Compound C

5 g of 2-methyl-4-(2,2-dicyanomethylene)chromone, 20 ml of toluene, 1.5 ml of piperdine, 1.5 ml of acetic acid and 7.8 g of 9-formyl-l-(1,1,7,7-tetramethyl)julolidine were placed in a 50 ml reaction vessel. The mixture was heated and refluxed for 18 hours. The mixture was then cooled to room temperature. After filtering, the mixture was washed with a small amount of toluene to obtain a product yield of 68%. Finally, the mixture was purified by sublimation. The melting temperature of the product is 252° C. ¹H-NMR: 8.88(1H, d, J=8.2 Hz), 7.73(1H, t, J=8.6 Hz), 7.43˜7.40(3H, m), 6.77(2H, br), 3.26(4H, t, J=5.8 Hz), 1.76˜1.61(4H, S), 1.25(12H, s) ppm ∘ Mass: 449(M+2) ∘ IR: 2203, 1624, 1585, 1550, 1476, 13120,1153, 769 cm^{31 1}^{_{∘}}

Formula of compound C is:

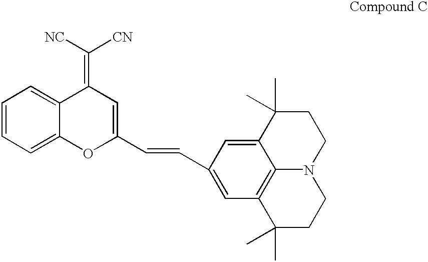

### COMPARATIVE EMBODIMENT 1

### DCM-1(4-(2,2-dicyanomethylene)-2-methyl-6(p-dimethylaminostyrl)-4H-pyrane)

224 mg of 2,5-dimethyl-4-(2,2-dicyanomethylene)-4H-pyrane, 15 ml of toluene, 0.2 ml of acetic acid, 0.2 ml of piperdine and 236 mg of 4-(N,N-dimethyl)anlinealdehyde were 0o placed in a 50 ml reaction vessel. The mixture was heated and refluxed for 20 hours. The mixture was then cooled to room temperature. After filtering, the mixture was washed with a small amount of toluene to obtain a product yield of 74%. Finally, the mixture was purified by sublimation.

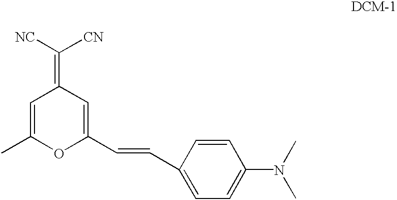

### COMPARATIVE EMBODIMENT 2

### DCM-2 (4- (2,2-dicyanomethylene)-2-methyl-6(p-julolidylstyrl)-4H-pyrane)

224 mg of 2,5-dimethyl-4-(2,2-dicyanomethylene)-4H-pyrane, 15 ml of toluene, 0.2 ml of acetic acid, 0.2 ml of piperdine and 315 mg of 9-formyl-julolidine were placed in a 50 ml reaction vessel. The mixture was heated and refluxed for 20 hours. The mixture was then cooled to room temperature. After filtering, the mixture was washed with a small amount of toluene to obtain a product yield of 58%. Finally, the mixture was purified by sublimation.

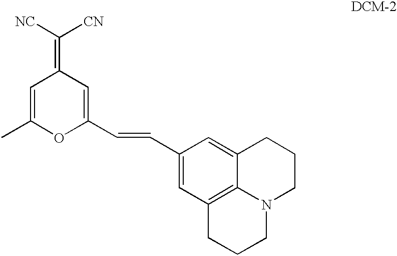

### COMPARATIVE EMBODIMENT 3

### DCJTB(4-(2,2-dicyanomethylene)-2-t-butyl-6(p-(1,1,7,7-tetramethyl)julolidystyrl-4H-pyrane)

224 mg of 2-methyl-5-t-butyl-4-(2,2-dicyanomethylene)-4H-pyrane, 15 ml of toluene, 0.2 ml of acetic acid, 0.2 ml of piperdine and 348 mg of 9-formyl-1-(1,1,7,7-tetramethyl)julolidine were placed in a 50 ml reaction vessel. The mixture was heated and refluxed for 18 hours. The mixture was then cooled to room temperature. After filtering, the mixture was washed with a small amount of toluene to obtain a product yield of 79%. Finally, the mixture was purified by sublimation.

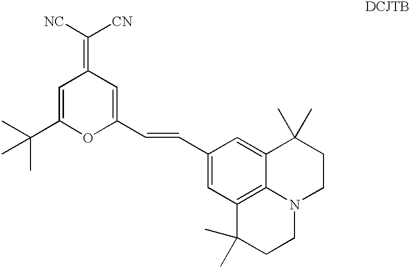

The following embodiments are carried out using the compounds synthesized above to fabricate the organic electroluminescent devices. Each device includes layers of hole-injection layer, hole-transport layer, light emitting layer and electron-transport layer.

### EMBODIMENT 4

Fabrication of the EL Device Using Compound A

An Indium-tin-oxide coated glass substrate (anode substrate) was sequentially washed in a cleaning solution, rinsed in de-ionized water and dried. Copper phthalocyanine was vapor deposited onto the ITO glass as the hole-injection layer (150 Å). Onto the hole-injection layer, a hole-transporting layer material N,N′-bis-(1-naphthyl)-N,N′-diphenylbenzidine (600 Å) was again vapor deposited. Next, onto the hole-transporting layer, the main host alumium-tris-8-hydroxyquinoline and compound A 2% (v/v) (150 Å) as the guest were co-deposited to become the light emitting layer.

Subsequently, alumium-tris-8-hydroxyquinoline (350 Å) was vapor deposited onto the light emitting layer as the electron-transporting layer. Mg—Ag alloy was then vapor deposited onto the electron-transporting layer as the anode. The element was then packaged in a dry glove box full of nitrogen for protection against ambient environment.

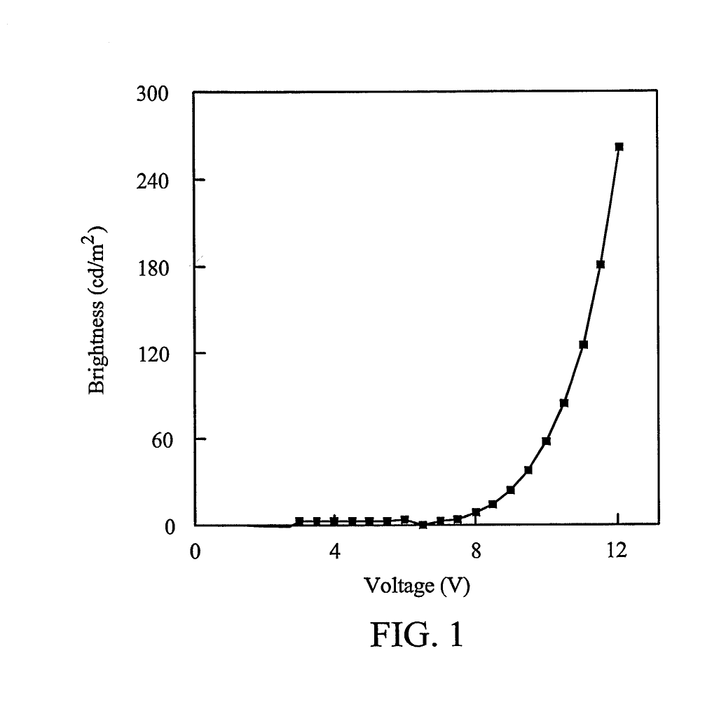

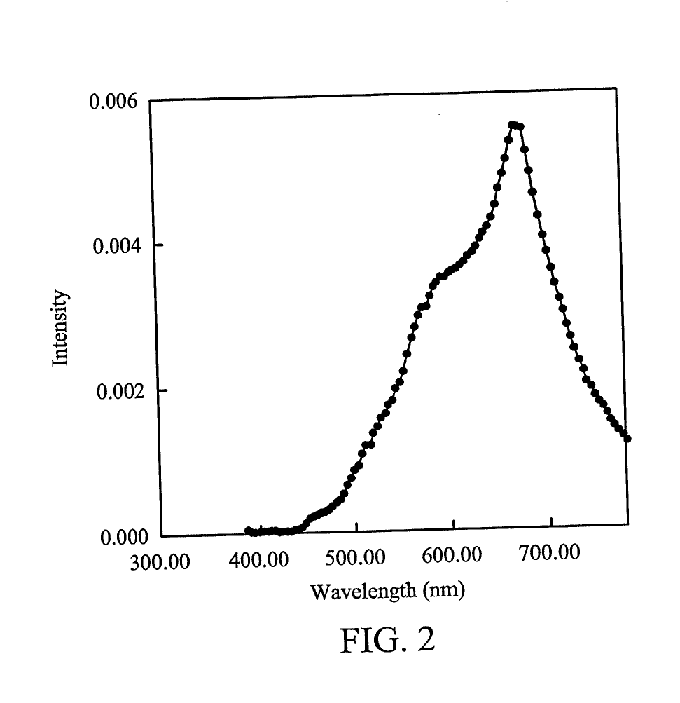

The organic EL device obtained in the above embodiment was then tested for its maximum wavelength λₘₐₓ in the EL spectra and CIE coordinate. The result is shown in Table 1. It is found that the CIE coordinate and wavelength are very close to NTSC standards: wavelength=650 nm and CIE coordinate x=0,67, y=0.33. The brightness and voltage were then plotted as FIG. 1. FIG. 2 shows the intensity vs wavelength of the obtained EL device.

### EMBODIMENT 5

Fabrication of the EL Device Using Compound B

An Indium-tin-oxide coated glass substrate (anode substrate) was sequentially washed in a cleaning solution, rinsed in de-ionized water and dried. Copper phthalocyanine was vapor deposited onto the ITO glass as the hole-injection layer (150 Å). On the hole-injection layer, a hole-transporting layer material N,N′-bis-(1-naphthyl)-N,N′-diphenylbenzidine (600 Å) was again vapor deposited. Next, on the hole-transporting layer, the main host alumium-tris-8-hydroxyquinoline and compound B 2% (v/v) (150 Å) as the guest were both vapor deposited to become the light emitting layer.

Subsequently, alumium-tris-8-hydroxyquinoline (350 Å) was vapor deposited onto the light emitting layer as the electron-transporting layer. Mg—Ag alloy was then vapor deposited onto the electron-transporting layer as the anode. The element was then packaged in a dry glove box full of nitrogen for protection against ambient environment.

The organic EL device obtained in the above embodiment was then tested for its max wavelength λₘₐₓ in the EL spectra and CIE coordinate. The result is shown in Table 1.

### EMBODIMENT 6

Fabrication of the EL Device Using Compound C

An Indium-tin-oxide coated glass substrate (anode substrate) was sequentially washed in a cleaning solution, rinsed in de-ionized water and dried. Copper phthalocyanine was vapor deposited onto the ITO glass as the hole-injection layer (150 Å). On the hole-injection layer, a hole-transporting layer material N,N′-bis-(1-naphthyl)-N,N′-diphenylbenzidine (600 Å) was again vapor deposited. Next, on the hole-transporting layer, the main host alumium-tris-8-hydroxyquinoline and compound C 2% (v/v) (150 Å) as the guest were both vapor deposited to become the light emitting layer.

Subsequently, alumium-tris-8-hydroxyquinoline (350 Å) was vapor deposited onto the light emitting layer as the electron-transporting layer. Mg—Ag alloy was then vapor deposited onto the electron-transporting layer as the anode. The element was then packaged in a dry glove box full of nitrogen for protection against ambient environment.

The organic EL device obtained in the above embodiment was then tested for its max wavelength A max in the EL spectra and CIE coordinate. The result is shown in Table 1.

### COMPARATIVE EMBODIMENT 4

Fabrication of the EL Device Using DCM-1

An Indium-tin-oxide coated glass substrate (anode substrate) was sequentially washed in a cleaning solution, rinsed in de-ionized water and dried. Copper phthalocyanine was vapor deposited onto the ITO glass as the hole-injection layer (150 Å). On the hole-injection layer, a hole-transporting layer material N,N′-bis-(1-naphthyl)-N,N′-diphenylbenzidine (600 Å) was again vapor deposited. Next, on the hole-transporting layer, the main host alumium-tris-8-hydroxyquinoline and DCM-1 1% (v/v) (150 Å) as the guest were both vapor deposited to become the light emitting layer.

Subsequently, alumium-tris-8-hydroxyquinoline (350 Å) was vapor deposited onto the light emitting layer as the electron-transporting layer. Mg—Ag alloy was then vapor deposited onto the electron-transporting layer as the anode. The element was then packaged in a dry glove box full of nitrogen for protection against ambient environment.

The organic EL device obtained in the above embodiment was then tested for its max wavelength λₘₐₓ in the EL spectra and CIE coordinate. The result is shown in Table 1.

### COMPARATIVE EMBODIMENT 5

Fabrication of the EL Device Using DCM-2

An Indium-tin-oxide coated glass substrate (anode substrate) was sequentially washed in a cleaning solution, rinsed in de-ionized water and dried. Copper phthalocyanine was vapor deposited onto the ITO glass as the hole-injection layer (150 Å). On the hole-injection layer, a hole-transporting layer material N,N′-bis-(1-naphthyl)-N,N′-diphenylbenzidine (600 Å) was again vapor deposited. Next, on the hole-transporting layer, the main host alumium-tris-8-hydroxyquinoline and DCM-2 1% (v/v) (150 Å) as the guest were both vapor deposited to become the light emitting layer.

Subsequently, alumium-tris-8-hydroxyquinoline (350 Å) was vapor deposited onto the light emitting layer as the electron-transporting layer. Mg—Ag alloy was then vapor deposited onto the electron-transporting layer as the anode. The element was then packaged in a dry glove box full of nitrogen for protection against ambient environment.

The organic EL device obtained in the above embodiment was then tested for its max wavelength A max in the EL spectra and CIE coordinate. The result is shown in Table 1.

### COMPARATIVE EMBODIMENT 6

Fabrication of the EL Device Using DCJTB

An Indium-tin-oxide coated glass substrate (anode substrate) was sequentially washed in a cleaning solution, rinsed in de-ionized water and dried. Copper phthalocyanine was vapor deposited onto the ITO glass as the hole-injection layer (150 Å). On the hole-injection layer, a hole-transporting layer material N,N′-bis-(1-naphthyl)-N,N′-diphenylbenzidine (600 Å) was again vapor deposited. Next, on the hole-transporting layer, the main host alumium-tris-8-hydroxyquinoline and DCJTB 0.5% (v/v) (150 Å) as the guest were both vapor deposited to become the light emitting layer.

Subsequently, alumium-tris-8-hydroxyquinoline (350 Å) was vapor deposited onto the light emitting layer as the electron-transporting layer. Mg—Ag alloy was then vapor deposited onto the electron-transporting layer as the anode. The element was then packaged in a dry glove box full of nitrogen for protection against ambient environment.

The organic EL device obtained in the above embodiment was then tested for its max wavelength λₘₐₓ in the EL spectra and CIE coordinate. The result is shown in Table 1.

TABLE 1

| | |
|---|---|
| max wavelength | |
| λₘₐₓ (nm) | CIE (x, y) |

the present invention

| | | |
|---|---|---|
| compound A | 670 | 0.66, 0.33 |
| compound B | 630 | 0.66, 0.36 |
| Compound C | 660 | 0.66, 0.34 |

prior art

| | | |
|---|---|---|
| DCM-1 | 610 | 0.62, 0.36 |
| DCM-2 | 640 | 0.64, 0.36 |
| DCJTB | 620 | 0.62, 0.37 |

From Table 1, it is obvious that the material for red organic EL devices provided in the present invention shows improved CIE compared to the material used in the prior art. CIE coordinate and wavelength both conform with NTSC standards (max wavelenth λₘₐₓ650 nm; CIE coordinate x=0.67, y=0.33). It is also observed that the red color EL of the devices using the novel compound provided in this invention appears deeper and more saturated. In addition, the material provided by the present invention is easy to synthesize. Consequently, product yield is increased and product costs are lowered.

The foregoing description of the preferred embodiments of this invention has been presented for purposes of illustration and description. Obvious modifications or variations are possible in light of the above teaching. The embodiments were chosen and described to provide the best illustration of the principles of this invention and its practical application to thereby enable those skilled in the art to utilize the invention in various embodiments and with various modifications as are suited to the particular use contemplated. All such modifications and variations are within the scope of the present invention as determined by the appended claims when interpreted in accordance with the breadth to which they are fairly, legally, and equitably entitled.

What is claimed is:

1. A compound, as shown in the following formula: 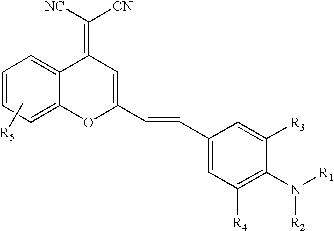
wherein, R₁ and R₂ are individually hydrogen, alkyl of from 1 to 20 carbon atoms, aryl, carbocyclic and other heterocyclic system, and R₃ and R₄ are individually hydrogen, alkyl of from 1 to 10 carbon atoms, and a branched or unbranched 5 or 6 member substituent ring connecting with R₁ and R₂ respectively; and R₅ is hydrogen, alkyl of from 1 to 10 atoms and a 5 or 6 member carboncyclic and other heterocyclic system connecting with benzenic ring; but when R₃₋₅ have no substitution groups present, R₁ and R₂ are hydrogen, alkyl of 2 to 20 carbon atoms, aryl, carbocyclic and other heterocyclic system.

2. The compound as claimed in claim 1, wherein R₁ and R₂ are methyl, ethyl, propyl, n-butyl, —(CH₂)₄—, —(CH₂)₅—, aryl, such as phenyl, furyl, thienyl, pyridyl or other heterocyclic system; R₃ and R₄ are hydrogen, methyl, ethyl, propyl, n-butyl, i-propyl, t-butyl, sec-butyl, t-amyl and —(CH₂)₃—, —(CH₂)₄—, or heteroaryl, including phenyl furyl, thienyl, pyridyl and other heterocyclic system connecting with benzenic ring.

3. An organic electroluminescent element, containing the compound as claimed in claim 1.

4. An organic electroluminescent element, containing the compound as claimed in claim 2.

5. An organic electroluminescent device, comprising an anode, a cathode, and an electroluminescent element as claimed in claim 3.

6. An organic electroluminescent device, comprising an anode, a cathode, and an electroluminescent element as claimed in claim 4.
